(19) United States
(12) Patent Application Publication
(10) Pub. No.: US 2004/0191330 A1
(43) Pub. Date: Sep. 30, 2004

(54) Daily skin care regimen

(76) Inventors: Candace R. Keefe, Dove Canyon, CA (US); Antoaneta Abbruzzese, Aliso Viejo, CA (US); Elizabeth A. Vervynck, Laguna Beach, CA (US); Pierre Bottiglieri, Conthey Sion (CH)

Correspondence Address: KENNETH J. HOVET, NORDMAN, CORMANY, HAIR & COMPTON, P.O. BOX 9100, 1000 TOWN CENTER DRIVE, OXNARD, CA, 93031-9100, US

(21) Appl. No.: 10/404,174

(22) Filed: Mar. 31, 2003

(51) Int. Cl. A61K 9/127; A61K 33/34; A61K 38/08

(52) U.S. Cl. 424/638; 514/17; 424/450

(57) ABSTRACT

A skin treatment regimen comprising six distinct products including a hydrating wash, a toner-astringent, an eye cream, a facial serum having alpha hydroxy and Beta Hydroxy Acids, a day cream having a sunscreen agent, and a night cream having spent grain wax and Glycine Soja (Soybean) Protein. The regimen includes a five step morning process comprising washing the face with the hydrating wash, applying the toner-astringent, applying the eye cream to an eye area of the face, applying the facial serum to the face, and applying the day cream. The regimen further includes a five step process in the evening prior to going to bed, comprising washing the face with the hydrating wash, applying the toner-astringent, applying the eye cream to an eye area of the face, applying the facial serum, and applying the night cream.

### BACKGROUND OF THE INVENTION

1. Field of the Invention

The present invention pertains generally to skin care products. More particularly, the invention relates to skin care preparations and regimens for applying skin care products sequentially to maximize the therapeutic skin care effect.

2. Description of Related Art

The skin is a major bodily organ that performs a variety of functions, including the maintenance of moisture within the body. The skin also acts as a barrier to foreign objects, sunlight, chemical pollutants, and pathogens, all of which can have deleterious effects on the body.

The structure of skin: Skin comprises essentially three layers—the epidermis, the dermis, and the subcutaneous tissue. The epidermis is comprised of four sub-layers. The stratum corneum, the outermost layer of the epidermis, is composed of flat, dead keratinized cells (corneocytes) which have lost their nuclei, but possess high levels of keratin, a horny protein found in hair and fingernails, giving the outermost layer of the epidermis a toughness which resists abrasion and injury. These corneocytes are embedded in a bilayer matrix of lipids known as ceramides. The combination of the lipid matrix and the keratinized cells helps create an excellent moisture barrier that resists dehydration of the subject. Without this moisture barrier, bodily fluids would easily evaporate through the skin, and the subject would quickly die of dehydration. The dead cells of the stratum corneum are constantly being sloughed off through a slow exfoliation through normal daily activity such as friction with clothing. When skin is too dry, epidermal hyperfoliation (scaly skin) can occur, as well as itching, and other undesirable symptoms of unhealthy or distressed skin.

The second layer of the epidermis is the stratum granulosum, a thin layer of epidermis, composed of only a few layers of cells below the stratum corneum. The cells of the stratum granulosum fill with keratin and die, thereby replacing the keratinized cells being sloughed off from the stratum corneum. The entire stratum corneum is replaced by new cells from the stratum granulosum over a period of approximately one month, depending upon the age of the subject and a variety of other factors.

The third layer of the epidermis is the stratum spinosim, and rests directly below the stratum granulosum.

The fourth layer of the epidermis is the stratum basale, the deepest layer of the epidermis. The stratum basale consists of a single row of columnar or cuboidal cells. Essentially all of the epidermal cells originate from this layer. As a result, new cells must be continually produced on this level. As the cells in this layer multiply, they are pushed upward to become part of the stratum granulosum, thereby continuing the cycle.

Keratinocytes, which are basale cells that produce the protein keratin, give the outer layer of skin its toughness as the keratinocytes migrate upward and die. Melanocytes are basale cells that produce melanin, the light absorbing substance that gives skin its color, and protects skin against ultraviolet radiation. A single melanocyte has multiple dendritic fingers that extend out and connect to keratinocytes.

Typically, an “epidermal melanin unit” comprises a single melanocyte linked by its dendritic fingers to about thirty six keratinocytes. Each melanocyte has organelles (miniature organs) known as “melanosomes” that synthesize melanin. The melanosomes are directed through the dendrites of the melanocyte by means of the Golgi system, and then by “motor proteins” along a network of microtubules until reaching a keratinocyte, thereby pigmenting the keratinocytes within its epidermal melanin unit. Elliptically shaped melanocytes produce a brown to black pigment, and spherically shaped melanocytes produce a red-yellow pigment. The ratio of melanocytes to keratinocytes within an “epidermal melanin unit” does not differ by race or skin color.

Stimulation of melanogenesis (the production of melanin) involves an increase in intracelllular cyclic AMP levels, which in turn regulate protein kinase PKC-b activity. Receptors on the surface of melanocytes are stimulated by melanocyte-stimulating hormone (MSH), which increases in response to bombardment from UV (ultraviolet) light. UV light is damaging to the skin, and is believed to be the leading cause of skin cancer including melanoma. Because the production of melanin is increased in response to UV bombardment, an increase in the production of melanin or darkening of the skin can be evidence that some damage to the skin has recently occurred.

The dermis, or corium, the second major layer of skin, is typically four to nine times as thick as the epidermis. Melanocytes begin migrating from the dermis to the epidermis by the eighth week of life, and the migration is normally completed by birth. Because the dermis usually lacks melanocytes or keratinocytes, it also lacks the melanin pigment or the keratin levels of the epidermis. The dermis is largely formed of collagen, a triple twine helical protein, which forms the greater portion of the structural network of the dermis, giving it the strength and durability. Elastin is another fibrous structure in the dermis. It is similar to collagen, but is more stretchable, giving the skin greater elasticity and an ability to restore to an original shape.

Fibroblasts, spindle-shaped cells with cytoplasmic processes present in connective tissue, are responsible for forming collagen fibers and elastin fibers. An inactive fibroblast is sometimes called a fibrocyte. Although both elastin and collagen proteins are part of the structural extra-cellular matrix (ECM) of the dermis, the elastin contributes more to the actual structural integrity of the matrix, and the collagen is more responsible for building scar tissue to mend cuts and abrasions. With age, the skin's collagen and elastin deteriorate and break down, causing the skin to become thinner, resulting in sagging and wrinkling.

The spaces within the fibrous collagen/elastin matrix are filled by marcomolecules known as proteoglycans and glycosaminoglycans (GAGs). Together, these glycans form a water saturated gel in which water, water soluble molecules, ions, hormones and peptides can circulate. These water saturated glycans within the extra-cellular matrix (ECM) help to hold the matrix up, much as if the glyeans were gelatin, and the collagen/elastin extra-cellular matrix were baffles and channels within a water bed. If the collagen/elastin matrix dehydrates, loses its ability to retain moisture, or is decreased in quantity, the matrix collapses. Since there is a maximum weight ratio of water to proteoglycan, if the quantity of proteoglycan is reduced, the water retention is also reduced. Alternatively, if deterioration of the glycan gel impedes cells from migrating through the glycan gel, or if mitosis is impeded, cellular replenishment is hindered.

With age, the number of fibroblasts, mastocytes and blood vessels are reduced. As a result, deterioration of the glycan gel or deterioration of the collagen/elastin matrix can cause a downward spiral. The tone, quality and elasticity of the skin notably deteriorates with age. Collagen production is decreased, the dermis atrophies, and the skin wrinkles and sags.

The replenishment of the dermis is governed by a variety of factors, including growth hormone (GH) and insulin growth factors (IGF). These hormones are produced by the pituitary and hypothalamus, and tend to decrease with age, leading to a slowing of cellular regeneration.

The dermis also contains sebaceous glands, which generally lie adjacent to hair follicles and are connected to the hair follicle by a short duct. The sebaceous gland secretes sebum (oil) into the hair follicle, providing a lubricant for the hair and skin. Sebum is a semifluid substance composed of waxes, fatty acids, cholesterol, and debris from skin cells. As a result, sebum helps to sequester moisture within the skin and body, preventing dehydration and keeping the skin and hair glossy and the skin pliable. Sebum is also integral to the formation of vitamin D and is hostile to certain forms of harmful bacteria. Sebum produced in the dermis is, therefore, important to the health of the epidermis as well.

The dermis also has nerve endings and blood vessels which supply nutrients to the epidermis. As a result, the skin quality of the epidermis depends on the quality of the interface between the epidermis and the dermis. The loss of adhesion between these layers is, therefore, destructive to the long term health, and the youthful feel and appearance, of the epidermis.

A variety of sensory receptors are also located in the skin. These include Pacinian corpuscles (for sensing motion and orientation of body parts), Meissner's corpuscles (touch sensors), Ruffini corpuscles (detecting hot, cold, pressure, and joint rotation), and Merkel's disks (sensing continuous touch, but probably not rising to the level of pressure).

Beneath the dermis is a fatty subcutaneous layer or subcutis, also known as the hypodermis. Sweat glands or “eccrine glands” are commonly located in the upper regions of the hypodermis, just beneath the dermis. Sweat glands are regulated by signals from the hypothalamus region of the brain to secrete moisture for regulating body temperature and cooling the body. Sweat ducts carry the moisture from the sweat gland, extending through the dermis and epidermis, terminating as sweat pores on the surface of the skin.

Free radicals: Free radicals are produced in a variety of ways, including through metabolic processes, and are, therefore, not limited to attacking the outer epidermal layers. Free radicals are positively charged molecules comprising an unpaired electron. In attempting to find a paired electron, free radicals scavenge electrons from other atoms or molecules (oxidation). An initial free radical can produce a cascading chemical reaction that produces a chain of free radicals, each stealing an electron from the next molecule. This process does not direct itself to any productive metabolic function. Rather, it is a destructive chemical chain reaction that damages the subjects body on a cellular level. Every person has billions of free radicals floating about in their body. Although free radicals can attack any part of a cell, because the cell membrane is composed primarily of easily oxidized fatty acids, it is a primary target for damage by free radicals. The cell membrane is a highly sophisticated and selective barrier that controls what comes in and goes out of a cell. Within a cell are sub-cellular components known as organelles. Each organelle is also wrapped in its own protective membrane that is subject to damage from free radicals. Mitochondria is the organelle specialized to convert food and oxygen into energy. Lysosomes scavenge waste within a cell, sometimes breaking down a protein into amino acids for recycling, sometimes processing a molecule for disposal if it is not fit for recycling.

The cell nucleus is also an organelle that houses the DNA blueprint of the cell. As noted, the cell membrane; and the protective membranes of each of the sub-cellular components are highly susceptible to damage from free radicals. This damage compromises the ability of the cell, or organelle, to perform its function properly, affecting energy production, transporting of nutrients, waste disposal, or even mutating the core DNA, causing cancer or other cellular abnormalities. As a result, free radicals are believed to be a key element in accelerating the aging process, contributing to the loss of skin firmness, elasticity, and general health and appearance.

Sunlight: When a stable molecule is broken apart into two component parts, the destruction can produce one or more molecules having an unpaired electron, thereby forming free radicals. However, in order to break a stable molecule apart, an input of energy must overcome the Gibbs energy of the various bonds holding the molecule together. Low energy radiation, such as radio or infra-red light, typically does not have the energy to break a molecule apart and contribute to the formation of free radicals. However, waveforms in the higher energy regions of the spectrum, such as ultraviolet radiation, are more likely to possess sufficient energy to break molecules apart into free radicals that can damage cells. Molecular damage can also include direct damage to the DNA within a cell's nucleus. A harmful mutation may result that can lead to cancer or other deleterious effects on the skin. Although some harmful radiation can be produced through indoor lighting, commercially produced light strives for economic efficiency. This means that it attempts to minimize the production of light falling in the invisible ranges of the electromagnetic spectrum, including the lower energy infra-red wavelengths, and the higher energy ultraviolet wavelengths. Natural sunlight, however, is not restrained by economic considerations, and produces a broad spectrum of radiation, including low energy radiation below the visible spectrum, and high energy radiation above the visible spectrum. The ultraviolet light produced by the sun is the most damaging to the skin.

Although the epidermis is the outermost layer of skin and, therefore, most exposed to sunlight and chemical pollutants, the aging process is destructive of all epidermal layers as well as the dermal layer. This is due to a number of factors. First, sunlight is not completely stopped by the stratum corneum, the outer layer of the epidermis, but can penetrate to the basel cell level of the epidermis, and even into the dermis, thereby damaging inner regions of the skin. Additionally, although the stratum corneum can help resist chemical pollutants, many toxic chemicals penetrate the stratum corneum and irritate or damage deeper layers of the epidermis and dermis. Contact dermatitis, for example, is less common on the palms of the hands, where the stratum corneum is thicker, thereby more effectively resisting penetration of toxic pollutants. Finally, as noted, antioxidants are produced through the metabolic process as well as from ultraviolet radiation and other external sources, thereby bombarding the skin cells with damaging antioxidants from within.

U.S. Pat. No. 4,272,544 to Cella et al. (“Cella '544”) is a process directed to the application and use of four products disclosed in related patents which were mutually and cooperatively incorporated by reference, including U.S. Pat. No. 4,278,570 to Flom (“Flom 570”) which is directed to “a skin cleanser formulation, U.S. Pat. No. 4,268,502 to Martin (“Martin '502”) which is directed to a Skin Tonic, U.S. Pat. No. 4,268,526 to Vargas et al. (“Vargas '526”) which is directed to a skin cream, and U.S. Pat. No. 4,272,519 to Herrold et al. (“Herrold”) which is directed to a Skin Lotion formulation. Cella reviews the chemical formulas for each of the above skin care products and discloses that the preferred regimen usually begins in the morning with the use of a tonic, followed by a lotion. Later, usually at night, a cleanser is used, and then the tonic and cream are applied. The order and timing of use of the four products can be varied to suit individual needs, and some effect will be obtained even if all the compositions are not used or if there is a delay between usages. However, the desired effect is primarily to enhance exfoliation of dead skin cells. Neither Cella '544 nor the related corporate patents of Flom '570, Vargas '526, Martin '502 and Herrold '519 teach or suggest an eye cream used in conjunction with a specified regimen. Neither do the above references teach or suggest the enhancement of lipolysis in select facial areas, nor the suppression of melanogenesis, the protection of skin from UV radiation, the enhancement of the moisture barrier in the stratum corneum of the epidermis, the nourishment of the cell membrane, the enhancement of production of collagen and elastin, the scavenging of free radicals and a wide variety of other important skin care functions.

### SUMMARY OF THE INVENTION

The present invention overcomes many of the prior art omissions by providing a skin care regimen that includes an eye cream for treating facial skin surrounding one's eyes. The eye cream may include pentapeptides; Alpha-Lipoic Acid; magnesium ascorbyl phosphate; Kojic Acid; copper; vitamin K; a Bio-Hydria® complex comprising a bio-herbal blend of members selected from a group consisting of comfrey, cucumber, birch leaf, watercress, clover, ginseng and St. John's wort; and a nanospheres complex, each nanosphere comprising gamma linoleic acid (GLA) within a phospholipid membrane. The pentapeptides within the eye cream advantageously define a LYS-THR-THR-LYS-SER chemical structure. The nanosphere complex may further include vitamins and/or esters of vitamins A, E and C dissolved with the gamma linoleic acid. The eye cream may advantageously include Morus Bombycis Root Extract and Arctostaphylos Uva Ursi Leaf Extract. As used herein, the term “vitamin” includes esters such as palmitate and acetate, complexes such as magnesium ascorbyl phosphate, and other compounds known to act as precursors, derivatives or substitutes for vitamins.

The skin care regimen further includes a hydrating wash for use in cleansing the facial skin prior to application of the eye cream. The hydrating wash utilizes nanospheres comprising gamma linoleic acid (GLA) and vitamins within a phospholipid membrane. The wash also includes effective amounts of magnesium ascorbyl phosphate; Alpha-Lipoic Acid; and at least one cleansing agent. The cleansing agents can be selected from a group consisting of sodium lauroamphoacetate, sodium trideceth sulfate, disodium laureth sulfosuccinate and disodium lauroamphoacetate.

The skin care regimen also includes a toner cleanser having effective amounts of the following: nanospheres comprising gamma linoleic acid (GLA) and vitamins within a phospholipid membrane; magnesium ascorbyl phosphate; Alpha-Lipoic Acid; a Bio-Hydria® complex comprising a bio-herbal blend of members selected from a group consisting of comfrey, cucumber, birch leaf, watercress, clover, ginseng and St. John's wort; and hamamelis virginiana (witch hazel).

A serum is also part of the skin care regimen. The serum includes effective amounts of the above-described magnesium ascorbyl phosphate; Alpha-Lipoic Acid; a Bio-Hydria® complex comprising a bio-herbal blend of members selected from a group consisting of comfrey, cucumber, birch leaf, watercress, clover, ginseng, St. John's wort; and Alpha Hydroxy Acids; and Beta Hydroxy Acids.

Additionally, the skin care regimen includes a day cream having effective amounts of pentapeptides; Alpha-Lipoic Acid; a Bio-Hydria(® complex comprising a bio-herbal blend of members selected from a group consisting of comfrey, cucumber, birch leaf, watercress, clover, ginseng and St. John's wort; nanospheres comprising gamma linoleic acid (GLA) and vitamins within a phospholipid membrane; magnesium ascorbyl phosphate; and a sunscreen agent. The sunscreen agent is selected from among a group consisting of octinoxate and oxybenzone.

Still further, the skin care regimen includes a night cream having effective amounts of pentapeptides; Alpha-Lipoic Acid; a Bio-Hydria® complex comprising a bio-herbal blend of members selected from a group consisting of comfrey, cucumber, birch leaf, watercress, clover, ginseng and St. John's wort; nanospheres comprising gamma linoleic acid (GLA) and vitamins within a phospholipid membrane; magnesium ascorbyl phosphate; spent grain wax; and Glycine Soja (Soybean) Protein.

The regimen provides a series of skin treatment steps beginning with the step of washing one's face with the above-described hydrating wash. Subsequently, application of the toner-cleanser may be used, which is followed with the above-referenced facial serum. Thereafter, the above referenced eye cream is applied, which may be followed by application of day cream or night cream as appropriate.

A toner-astringent having hamamelis virginiana (witch hazel) may optionally be used subsequent to the step of applying the hydrating wash and prior to the step of applying the eye cream. The day cream preparation includes effective amounts of sunscreen and a pentapeptide having a LYS-THR-THR-LYS-SER chemical structure. Because of its sunscreen qualities, the day cream is applied in the morning subsequent to the application of the facial serum.

The regimen advantageously further provides a sequence of skin treatment steps performed in the evening, including the steps of washing the face with the hydrating wash, applying the toner-astringent, applying the facial serum, applying the eye cream, and applying a night cream, wherein the night cream comprises spent grain wax, Glycine Soja (Soybean) Protein; and a pentapeptide having a LYS-THR-THR-LYS-SER chemical structure.

As mentioned, the morning regimen includes at least five skin treatment steps comprising washing the face with the hydrating wash; applying the toner-astringent to the face; applying the facial serum to the face; applying the eye cream to an eye area of the face; and applying the day cream to the face.

### DESCRIPTION OF THE PREFERRED EMBODIMENTS

The present invention is directed to morning and evening skin treatment regimens utilizing effective amounts of at least six defined products. To have the greatest effect, the six products are designed to be used in a coordinated sequential series of steps incorporating a multi-step morning regimen using at least five of the six products, and a multi-step evening regimen using at least five of the six products.

Key Ingredients:

Because many of the above products use commonly known ingredients, the following discussion is a summary of only select ingredients which uniquely enhance the effectiveness of the present skin care regimen. The names used for the ingredients herein include proprietary names, the contents of which are defined and terms derived from the International Nomenclature for Cosmetic Ingredients (INCI), as well as commonly used terms.

Antioxidants: The cascading production of free radicals, and their deleterious effects on skin was discussed in the background of the invention. Certain molecules, however, are able to “lend” an electron to a free radical without becoming free radials themselves. These chemicals are known as antioxidants. Studies have shown that they are effective in neutralizing free radicals when applied topically as well as when ingested. There are a variety of known antioxidants.

Alpha-Lipoic Acid is an antioxidant that penetrates the skin quickly and is efficiently absorbed into skin cells, making it an ideal antioxidant for cosmetic and therapeutic skin care.

Vitamin E: Vitamin E has long been recognized for its antioxidant properties. Additionally, vitamin E is believed to exhibit some anti-inflammatory properties by inhibiting the release of histamine and hydrolytic enzymes from mast cells and lysosomes, and by inhibiting the synthesis of Prostaglandin E₂, a substance believed to play an important role in the inflamation process.

Vitamin C and Magnesium Ascorbyl Phosphate, a water-soluble and stable vitamin C derivative. For some time it has been known that vitamin C exhibits antioxidant properties for neutralizing free-radicals which can damage skin, and that vitamin C promotes the formation of collagen.

Recent studies have also indicated that vitamin C in the form of magnesium ascorbyl phospate is absorbed per cutaneously by topical application. In addition to its antioxidant effects, it is an effective skin lightener, producing significant lightening effects in hyperpigmentation disorders and in lightening healthy unpigmented skin.

B-Group Vitamins: B group vitamins thiamine-HCL(B₁), riboflavin (B₂), pyridoxal-HCL, B12, Folic Acid, Nicotinamide, Ca-D-Pantothenate as well as vitamin E and biotin (vitamin H) are believed to stimulate cell respiration and fatty acid metabolism.

Copper is a mineral possessing some antioxidant properties. Additionally, copper bolsters enzymes that stimulate the growth of collagen, and stimulates elastin production and formation of cellular cement between cells. This helps to firm the skin. Copper is also believed to increase blood vessel formation and oxygenation within the skin. Although other copper compounds might be utilized, according to the preferred embodiment, the present invention envisions the use of copper gluconate.

L-Carnitine, Coenzyme A and Caffeine: Because of the synchronicity and the synergistic effect of these three compounds within present invention, they are discussed herein collectively. Adipocytes (fat cells) are found in the hypodermis, the third layer of skin resting directly beneath the dermis. This layer is found in most places of the body, including fat cells beneath the eyes. When fat is stored in the hypodermis beneath the eyes, it can form unsightly fatty deposits known as “bags.” The synergistic combination of L-Carnitine, Coenzyme A and Caffeine in a topical application is configured to metabolize the subcutaneous fat. It is particularly suited for use underneath a subject's eyes, thereby reducing unsightly “bags” beneath the eyes.

Adipocytes are comprised largely of triglycerides, which form the body's energy reserves. Therefore, the first step in lipolysis (the metabolization of fat) begins with the degradation of triglycerides into free fatty acids (FFA) and glycerol. This reaction occurs in the cytosol of the adipocytes. From there, the long chain of free fatty acids are directed into the interior of the mitochondria, the organelle within the cell responsible for “burning” cellular fuel.

The transportation of FFAs across the mitochondrial membrane occurs in distinct steps. First, the FFAs are transformed by binding to a coenzyme A molecule (CoA). This forms an Acyl-CoA structure. Next, the enzyme carnitine-acyl-transferase-I binds to the Acyl group of carnitine, thereby forming acyl-carnitine, the only fatty acid capable of crossing the mitochondrial membrane. After acyl-carnitine is formed, the CoA molecule or a chemical precursor of CoA is again available to repeat this process.

Within the mitochondria, carnitine acyl transferase II unbinds the acyl group from carnitine to transfer it to an intra-mitochondrial coenzyme A molecule. Once this step is completed, the carnitine is released by the mitochondrion and is free to secure another fatty acid and escort it into the mitochondrion for burning. As a consequence, experimental evidence suggests that an intake of exogenous Coenzyme A and L-Carnitine will accelerate lipolysis (the burning of fat) and enhance the efficiency of the lipolytic process.

The process of lipolysis incorporates a number of chemical reactions not discussed herein. Integral to the lipolysis process, however, is cyclic nucleotide “cyclic AMP” or “cAMP.” However, cAMP can be transformed into a substantially useless form referenced as “AMP,” through the presence of phosphodiesterase, thereby hindering efficient lipolysis. To ensure the fat burning process to continue unimpeded, therefore, the influence of phosphodiesterase must be eliminated or inhibited. Caffeine has been proven to inhibit phosphodiesterase, thereby allowing the fat burning process to continue uninhibited. The synchronicity of these three compounds can be summarized in that coenzyme A breaks down triglycerides into burnable fatty acids, L-Carnitine transports the fatty acids into the cellular engines to be burned, and caffeine safeguards the efficiency of the process by inhibiting the effect of phosphodiesterase, thereby safeguarding the existence of the important nucleotide “cyclic AMP” (cAMP).

Lipid nanospheres are microscopic lipid bubbles that act as a delivery means for select ingredients, as well as the therapeutic value of the lipid bubble itself. Gamma linoleic acid (GLA) is an “essential” polyunsaturated fatty acid, that is, one that the body needs but cannot produce. Polyunsaturated fatty acids are important components of the skin lipid film that protects against dryness. As discussed above, it is the lipid barrier of the stratum corneum that resists water loss through the skin. When the lipid barrier is compromised, dehydration of the skin occurs, potentially damaging all of the layers of the skin.

Reinforcement of the lipid barrier reduces transepidermal water loss (TEWL), thereby increasing the softness and health of the skin. Borage oil, derived from the seed of a wildflower known as borago officinalis, is among the richest sources of gamma linoleic acid (GLA). Nanospheres are formed by mixing Borage oil, water and soy lecithin (phosolipids) in a highly pressurized environment. In this process, the oil disperses into ultra-small invisible oil droplets stabilized by a phospholipid membrane. Phospholipids with highly unsaturated fatty acids, such as gamma linoleic acid (GLA), are very mobile, and integrate well into the lamellar structure of the stratum corneum in the epidermis. When vitamins, including esters of vitamins A, C and E are dissolved in the GLA, the tiny spheres become an effective delivery device for the vitamins, as well as the therapeutic value of the polyunsaturated gamma linoleic acid itself.

Moreover, antioxidants vitamin C and vitamin E protect the body from various events related to the aging process, such as free radicals. Vitamin A exhibits a positive effect on skin aging and UV damage, reducing the depth of wrinkles and increasing the elasticity of the skin. Vitamin A also has effects similar to a growth hormone stimulating the production of skin cells. Embedding these vitamins in lipid nanospheres increases their penetration into the skin, and their effectiveness in reaching damaged areas of the skin and restoring or maintaining skin health. The net effects include, but are not limited to, smoothing and reducing wrinkles, increasing skin thickness, improving the moisture barrier of the stratum corneum, thereby preserving the skin's moisture, and restoring or preserving the elasticity and tone of the skin.

Spent grain wax is derived from spent barley grains produced in the brewing process during beer wort production. In wort production for brewing beer, barley is cleaned and watered, and germinate in about a week. The germination process is halted by heating the barley in a malt kiln. The malt is then crushed and fresh brewing water is added and warmed. The mixture degrades through an enzyme reaction into beer wort. At a predetermined point of degradation, the process is stopped, and the barley grains are removed and dried for extraction of lipophilic constituents. Spent grain wax is extracted through a supercritical carbon dioxide extraction process at sixty degrees centigrade in an oxygen free environment. Spent grain wax contains naturally occurring fatty acids, vitamins and phytosterols.

Commercially available spent-grain wax products include a variety of fatty acids, vitamins and photosterols, many of which occur naturally in the wax. These include “essential” fatty acids linoleic acid and linolenic acid, which are used by keratinocytes for the biosynthesis of membrane lipids, strengthening the cell membrane against damage from free-radicals and other damaging agents. Additionally, when acylated with linoleic acid, some ceramides act to strengthen the linkages between lipid bilayers and the corneocytes. This produces a more reliable water barrier in the epidermis, reducing the transepidermal water loss (TEWL), thereby maintaining skin moisture and health.

Non-essential fatty acids such as palmitic acid, myristic acid, stearic acid, and oleic acid, linoleic acid, linolenic acid, behenic acid, erucic acid and lignoceric acid also help form a more reliable epidermal permeability barrier. The presence of photosterols such as β-sitosterol in the spent grain wax further improve its effect of spent grain wax on supporting the epidermal barrier as well as exhibiting anti-pruritic effects. Because locally applied vitamins can reverse many of the degenerative processes related to skin aging, B group vitamins thiamine-HCL(B₁), riboflavin (B₂), pyridoxal-HCL, B12, Folic Acid, Nicotinamide, Ca-D-Pantothenate as well as vitamin E and biotin (vitamin H), which can occur naturally in spent grain wax, enhance the therapeutic effects even further, stimulating cell respiration and fatty acid metabolism.

The benefits of spent grain wax were first discovered as a result of noting that workers handling the barley malt during beer wort production had less itching on their hands, and the quality of their skin was improved through contact. As a result, the general therapeutic effects of spent grain wax have the important effect of reducing itching and skin irritation and generally improving the texture and feel of skin. This is particularly important in conjunction with the application of many of the hydroxy acids and ascorbic acid-type compounds that can have a drying or otherwise irritating effect on some skin.

Glycine Soja (Soybean) Protein: Glycine Soja (Soybean) Protein is a plant derivative known to contribute to the skin's softness, elasticity and smoothness.

Pentapeptides: As discussed above, the collagen and elastin forming the collagen/elastin extra-cellular matrix (ECM) in the dermis begin to break down in time, as well as the proteoglycans and glucosaminoglucans that fill the matrix. The pentapeptide LYS-THR-THR-LYS-SER, a fragment of the C-terminal portion of collagen I has been shown to enhance the synthesis of collagen I, collagen IV and fibronectin. However, the specific application of the LYS-THR-THR-LYS-SER pentapeptide is critical as studies have shown that different sequences can be statistically insignificant or even inhibitory. More significantly, it has been found that the LYS-THR-THR-LYS-SER pentapeptide is more effective on skin marked by a low density of fibroblasts, such as aging skin, which exhibits a reduced population of fibroblasts. The effect toward the neo-synthesis of collagen and fibronectin is even more effective when used in the presence of growth factor β-TGF. The net effect includes the production of collagen which in turn firms the skin and reduces winkles. Additionally, it has been found that LYS-THR-THR-LYS-SER pentapeptide stimulates the neosynthesis of glycosaminoglycans (GAGs), a substance that fills the collagen/elastin extra-cellular matrix (ECM). In summary, the referenced pentapeptide increases skin thickness and improves skin texture. It also reduces roughness, wrinkle volume and wrinkle depth by reinvigorating fibroblasts to produces significant quantities ofcollagen and glycosaminoglycans to restore the dermal matrix.

Kojic Acid: In various forms, Kojic Acid displays a variety of effects, including antibacterial action, antioxidant properties of sequestering heavy metal ions that accelerate oxidation, and lightening of the skin. Unlike many skin lighteners presently on the market, Kojic Acid does not lighten the skin by oxidation, but by impeding melanogenesis, the production of melanin within the melanocytes (melanin producing cells) located in the layer between the dermis and epidermis. Because Kojic Acid does not bleach or oxidize, but simply inhibits the production of pigment on a cellular level, Kojic Acid is not only milder and gentler to the skin than oxidation type lighteners, it is also more effective, particularly for melasma, or systemic types of hyperpigmentation, which respond less favorably to hydroquinone-type skin lighteners. The lightening effects of Kojic Acid can be expected to last only as long as the product is being used. When regular application is curtailed, melanocytes can re-commence pigmentation production.

As noted above, Kojic Acid also has some antioxidizing properties. While antioxidants are naturally beneficial in protecting the skin from free radicals, this antioxidant property further helps to preserve the freshness and effectiveness of any product containing Kojic Acid.

Alpha Hydroxy Acids and Beta Hydroxy Acids: Alpha Hydroxy Acids (AHAs) are water soluble non-toxic acids derived from foods, plants, lactic acid and citrus products. AHAs have been shown to break down select protein bonds, thereby promoting the shedding of dead cells and increasing cell proliferation. Skin treated by Alpha Hydroxy Acid can take on a smoother, more radiant look. AHAs are water soluble. Beta Hydroxy Acid (BHA) in the form of salicylic acid, is lipid (oil) soluble, but is also soluble in hot water and slightly soluble in water at room temperature. As a result, BHA is able to penetrate into the pore which contains sebum and exfoliate the dead skin cells that are built up inside the pore, and has antioxidant properties that can tighten and smooth skin.

Because of their complementary solubility, Beta Hydroxy Acid can sometimes be more effective when used in conjunction with oily skin, and AHAs can often be more effective when used in conjunction with less oily skin. When used together, they can represent a broad spectrum cosmetic skin treatment that effectively exfoliates dead skin cells, reduces brown spots and fine lines, and rejuvenates tissue.

Bio-Hydria® Complex is a proprietary term referring to a bio-herbal blend of members of a plant nutrient group that soften and condition skin, including Comfrey, Cucumber, Birch Leaf, Watercress, Clover, Ginseng; and St. John's wort. This bio-herbal blend provides a greater effect in maintaining beautiful and healthy skin than any of the component herbal ingredients in isolation.

The Six Products of the Present Invention:

The present invention includes six distinct skin care products utilizing the above ingredients, and many other ingredients as well. The following lists summarize the key ingredients of the six skin care products of the present invention, and offer a complete list of the ingredients to set forth the best mode of the invention, including recommended weight percentage ranges for select ingredients. Because the percentages are only a suggested range, the percentages suggested for any given product may not add up to exactly 100%. Additionally, some ingredients listed separately below are noted to be part of a blend. In such cases, the listed percent represents the percent of the that blend within a particular product. Therefore, the actual percentages of products comprising that blend is lower than the listed percentage. Moreover, those skilled in the art will recognize small differences in percentages are equivalent amounts envisioned within the spirit and scope of the claimed invention. In particular, it is expected that the weight percentages of at least the aforementioned key ingredients may vary about plus or minus 30%, depending on the user's objectives, age and skin condition.

Product 1: Hydratiny Cleanser:

The primary ingredients of the hydrating cleanser include lipid nanospheres with gamma linoleic acid (GLA) derived from borage oil, including vitamins and/or esters of vitamins A, C and E dissolved in the GLA, the borage oil preferably totaling from 0.01% to 4% of the Hydrating Cleanser by weight; Vitamin C in the form of Magnesium Ascorbyl Phosphate preferably totaling from 0.01% to 1.5% of the Hydrating Cleanser by weight; and Alpha-Lipoic Acid preferably totaling from 0.0003% to 0.2% of the Hydrating Cleanser by weight; and a cleansing blend of Sodium Lauroamphoacetate and Sodium Trideceth Sulfate preferably totaling from 1% to 13% of the Hydrating Cleanser by weight. The importance of combining the above compounds in a cleansing agent stems from the well known fact that cleansing agents, by their very nature, are not always well tolerated by the skin. The oil-removal feature of a cleanser can result in drying of the skin, and even skin irritation. By incorporating various protective agents, such as lipid nanospheres, in the cleansing process, the Hydrating Cleanser of the present invention overcomes shortcomings found in many alternative products.

The following is a listing for ingredients
of the Hydrating Cleanser:

| | | |
|---|---|---|
| Ascorbyl Palmitate | 0.050% | (part of a blend) |
| Borago Officinalis Seed Oil | 0.05% | (part of a blend) |
| Butylene Glycol | 0.1% | |
| Caprylic/Capric Triglyceride | 0.05% | (part of a blend) |
| Citrus Aurantium Dulcis (Orange) Oil | 0.6% | |
| Cocamidopropyl Betaine | 6.0% | |
| Disodium EDTA | 0.05% | |
| Disodium Laureth Sulfosuccinate | 3% | (part of a blend) |
| Disodium Lauroamphoacetate | 3.0% | (part of a blend) |
| Glycerin | 0.020% | (part of a blend) |
| Glycol Stearate | 1.5% | |
| Hexylene Glycol | 5.0% | (part of a blend) |
| Lecithin (various forms) | 0.02% | (part of a blend) |
| Magnesium Ascorbyl Phosphate | 0.050% | |
| Methylchloroisothiazolinone | 0.060% | (part of a blend) |
| Methylisothiazolinone | 0.060% | (part of a blend) |
| Panthenol | 0.020% | (part of a blend) |
| PEG-80 Sorbitan Laurate | 10% | |
| Phosphatidyl Choline | 0.02% | |
| Phospholipids | 0.010% | (part of a blend) |
| Polysorbate-20 | 0.6% | |
| Retinyl Palmitate | 0.050% | (part of a blend) |
| Sodium Cocoyl Isethionate | 27.0% | (part of a blend) |
| Sodium Lauroamphoacetate | 5.0% | |
| Sodium Trideceth Sulfate | 5.0% | (part of a blend) |
| Stearamine | 0.020% | (part of a blend) |
| Stearic Acid | 27% | (part of a blend) |
| Thioctic Acid | 0.001% | |
| Tocopheryl Acetate | 0.050% | (part of a blend) |
| Water | 40.949% | |

Product 2: Mist Balancing Toner:

The toner is comprised largely of Hamamelis Virginiana (witch hazel), an astringent that tightens and constricts body tissues, as well as some secondary cleansing effects, advantageously falling within the range of 10% to 72% of the Toner by weight. To ameliorate the potentially harsh or drying effects of witch hazel, other principal ingredients of the Mist Balancing Toner include lipid nanospheres with gamma linoleic acid (GLA) derived from borage oil, including vitamins and/or esters of vitamins A, C and E dissolved in the GLA, the borage oil advantageously falling within the range of 0.01% to 3.5% of the Toner by weight; Vitamin C in the form of Magnesium Ascorbyl Phosphate advantageously falling within the range of 0.003% to 1.5% of the Toner by weight; Alpha-Lipoic Acid advantageously falling in the range of 0.0003% to 0.2% of the Toner by weight, and a Bio-Hydria® complex derived from a bio-herbal blend including comfrey, cucumber, birch leaf, watercress, clover, ginseng and St. John's wort, the Bio-Hydriag complex advantageously falling within the range of 0.5% to 17% of the total weight of the Toner. In contrast to the many astringents on the market, this particular combination has been developed through testing to produce an astringent that is effective and yet, does not damage or dry out the skin.

The following is a listing for ingredients
of the Mist Balancing Toner:

| | | |
|---|---|---|
| Ascorbyl Palmitate | 0.010% | (part of a blend) |
| Borago Officinalis Seed Oil | 0.10% | (part of a blend) |
| Betula Alba Leaf Extract | 3.0% | (part of a blend) |
| Butylene Glycol | 3.0% | (part of a blend) |
| Caprylic/Capric Triglyceride | 0.10% | (part of a blend) |
| Citrus Aurantium Dulcis (Orange) | 0.1% | |
| Oil | | |
| Cucumis Sativus (Cucumber) | 3.0% | (part of a blend) |
| Fruit extract | | |
| Diazolidinyl Urea | 0.3% | |
| Glycerin | 3.0% | |
| Hamamelis Virginiana (Witch Hazel) | 30.00% | |
| Water | | |
| Hypericum Perforatum Extract | 3.0% | (part of a blend) |
| Lecithin (various forms) | 0.1% | (part of a blend) |
| Magnesium Ascorbyl Phosphate | 0.010% | |
| Methylparaben | 0.180% | |
| Nasturtium Officinale Extract | 3.0% | (part of a blend) |
| Panthenol | 0.10% | (part of a blend) |
| Panax Ginseng Root Extract | 3.0% | (part of a blend) |
| Phenoxyethanol | 0.5% | |
| Phosphatidyl Choline | 0.1% | (part of a blend) |
| Phospholipids | 0.010% | (part of a blend) |
| Polyamino Sugar Condensate | 3.0% | (part of a blend) |
| Polysorbate-20 | 1.0% | |
| Retinyl Palmitate | 0.010% | (part of a blend) |
| Sodium PCA | 3.3% | (part of a blend) |
| Stearamine | 0.10% | (part of a blend) |
| Symphytum Officinale Extract | 3.0% | (part of a blend) |
| Thioctic Acid | 0.001% | |
| Tocopheryl Acetate | 0.010% | (part of a blend) |
| Trifolium Pratense (Clover) | 3.0% | (part of a blend) |
| Flower Extract | | |
| Urea | 3.0% | (part of a blend) |
| Water | 61.689% | |

Product 3: Reactivating Facial Serum:

The primary ingredients of the Reactivating Facial Serum include Lipid nanospheres with gamma linoleic acid (GLA) derived from borage oil, including vitamins and/or esters of vitamins A, C and E dissolved in the GLA, the borage oil preferably falling within the range of 0.1% to 4% of the Facial Serum by weight; Vitamin C in the form of Magnesium Ascorbyl Phosphate, preferably falling in the range of 0.05% to 3.5% of the Facial Serum by weight; Alpha-Lipoic Acid preferably falling in the range of 0.0003% to 0.2% of the Facial Serum by weight; a Bio-Hydriae® complex including a bio-herbal blend derived from comfrey, cucumber, birch leaf, watercress, clover, ginseng and St. John's wort, the Bio-Hydriae complex preferably falling in the range of 0.1% to 13% of the Facial Serum by weight; Alpha Hydroxy Acid preferably falling in the range of 0.1% to 7.5%; and Beta Hydroxy Acid preferably falling in the range of 0.05% to 3% of the Facial Serum by weight. The specific combination of nanospheres and Bio-Hydria® complex are used to effectively ameliorate any drying effects that Alpha and Beta Hydroxy Acids may produce, thereby resulting in a facial serum superior to current products on the market.

The following is a listing for ingredients
of the Reactivating Facial Serum:

| | | |
|---|---|---|
| Allantoin | 0.10% | |
| Aloe Barbadensis Leaf Juice | 0.200 | |
| Ascorbyl Palmitate | 0.5% | (part of a blend) |
| Betula Alba Leaf Extract | 0.125 | |
| Bis-PEG-18 Methyl Ether Dimethyl Silane | 3.5% | |
| Borago Officinalis Seed Oil | 0.25% | (part of a blend) |
| Butylene Glycol | 1.0% | |
| Butylparaben | 0.050% | |
| Camellia Sinensis Leaf Oil/Extract | 2.0% | (part of a blend) |
| (green tea) | | |
| Caprylic/Capric Triglyceride | 0.25% | (part of a blend) |
| Citrus Aurantium Dulcis (Orange) | 2.0% | (part of a blend) |
| Oil/Extract | | |
| Citrus Medica Limonum (Lemon) | 2.0% | (part of a blend) |
| Fruit Extract | | |
| Cucumis Sativus (Cucumber) | 0.125% | |
| Fruit extract | | |
| Glycerin | 4.0% | |
| Hypericum Perforatum Extract | 0.125% | |
| Lactic Acid | 1.250% | |
| Lecithin | 0.25% | (part of a blend) |
| Magnesium Ascorbyl Phosphate | 0.2% | |
| Methylparaben | 0.25% | |
| Nasturtium Officinale Extract | 0.125 | |
| Panthenol | 0.20% | |
| Panax Ginseng Root Extract | 0.125% | |
| Phenoxyethanol | 0.75% | |
| Phosphatidyl Choline | 0.25% | |
| Phospholipids | 0.5% | (part of a blend) |
| Polyamino Sugar Condensate | 0.10% | (part of a blend) |
| Propylparaben | 0.10% | |
| Purus Malus (Apple) Fruit Extract | 2.0% | (part of a blend) |
| Retinyl Palmitate | 0.5% | (part of a blend) |
| Saccharum Officinarum (Sugar Cane) | 2.0% | (part of a blend) |
| Extract | | |
| Salicylic Acid | 0.100% | |
| Sodium Hyaluronate | 1.00% | |
| Sodium Lactate | 3.4% | |
| Sodium PCA | 0.125% | |
| Symphytum Officinale Extract | 0.125% | |
| Thioctic Acid | 0.010% | |
| Tocopheryl Acetate | 0.5% | (part of a blend) |
| Trifolium Pratense (Clover) | 0.125 | |
| Flower Extract | | |
| Urea | 0.1% | (part of a blend) |
| Water | 74.54% | |
| Xanthan Gum | 0.900% | |

Product 4: Corrective Eye Cream:

The primary ingredients of the Corrective Eye Cream includes the lipolysis enhancing combination of L-Carnitine, Coenzyme A and Caffeine for reducing fat deposits beneath the eyes, the combination preferably totaling from 0.003% to 1.5% of the weight of the Corrective Eye Cream; lipid nanospheres with gamma linoleic acid (GLA) derived from borage oil, including vitamins and/or esters of vitamins A, C and E dissolved in the GLA, the borage oil preferably totaling from 0.05% to 7.5% of the total weight of the Corrective Eye Cream; vitamin C in the form of Magnesium Ascorbyl Phosphate preferably totaling from 0.05% to 4.5% of the total weight of the Corrective Eye Cream; Alpha-Lipoic Acid preferably totaling from 0.0003% to 0.25% of the total weight of the Corrective Eye Cream; a Bio-Hydria® complex including a bio-herbal blend derived from comfrey, cucumber, birch leaf, watercress, clover, ginseng and St. John's wort, the Bio-Hydria® complex preferably totaling from 0.3% to 17.5% of the total weight of the Corrective Eye Cream; LYS-THR-THR-LYS-SER pentapeptides preferably totaling from 0.7% to 6.5% of the total weight of the Corrective Eye Cream; phytonadione (vitamin K) preferably totaling from 0.005% to 0.5% of the total weight of the Corrective Eye Cream; Kojic Acid preferably totaling from 0.002% to 0.5% of the total weight of the Corrective Eye Cream; Morus Bombycis Root Extract preferably totaling from 0.002% to 0.25% of the total weight of the Corrective Eye Cream; Arctostaphylos Uva Ursi Leaf Extract preferably totaling from 0.002% to 0.25% of the total weight of the Corrective Eye Cream; and copper gluconate preferably totaling from 0.002% to 0.25% of the total weight of the Corrective Eye Cream. This combination simultaneously enhances the growth of collagen and elastin, thereby firming the skin beneath the eyes, enhances the metabolization of fat deposits beneath the eyes, lightens the skin beneath the eyes, and otherwise enhances and protects the quality of the skin surrounding the eyes.

Although the preferred embodiment envisions the use of copper gluconate, other forms of copper and copper compounds, such as Alanine/Hystidine/Lysine/Polypeptide Copper HCL, Copper Aspartate, Copper PCA and Copper Tripeptide, are envisioned within the scope of the present invention.

The combination of the above referenced compounds, when used in conjunction with Polyamino Sugar Concentrate (sugar cane extract) produced the unexpected result of increased moisturization of the upper layer of the epidermis.

The following is a listing for ingredients
of the Corrective Eye Cream:

| | | |
|---|---|---|
| Allantoin | 0.1% | |
| Arctostaphylos Uva Ursi Leaf Extract | 0.01% | (part of a blend) |
| Ascorbyl Palmitate | 0.5% | (part of a blend) |
| Betula Alba Leaf Extract | 5.0% | (part of a blend) |
| Borago Officinalis Seed Oil | 0.25% | (part of a blend) |
| Butylene Glycol | 3.0% | |
| Caffeine | 0.01% | (part of a blend) |
| Camellia Sinensis Leaf Oil | 2.0% | |
| Caprylic/Capric Triglyceride | 0.25% | (part of a blend) |
| Carbomer (TEA carbomer) | 3.0% | |
| Carnitine | 0.01% | (part of a blend) |
| Ceteareth-20 | 1.0% | (part of a blend) |
| Cetearyl Alcohol | 1.0% | (part of a blend) |
| Cetyl Alcohol | 1.0% | |
| Citrus Aurantium Dulcis (Orange) | 0.15% | |
| Oil/Extract | | |
| Coenzyme A | 0.01% | (part of a blend) |
| Copper Gluconate | 0.01% | |
| Cucumis Sativus (Cucumber) | 5.0% | (part of a blend) |
| Fruit extract | | |
| Decyl Oleate | 2.0% | |
| Diazolidinyl Urea | 0.4% | |
| Glycerin | 2.0% | |
| Glyceryl Stearate | 4.0% | (part of a blend) |
| Glycyrrhiza Glabra (Licorice) | 0.01% | (part of a blend) |
| Root Extract | | |
| Hypericum Perforatum Extract | 5.0% | (part of a blend) |
| Kojic Acid | 0.01% | (part of a blend) |
| Lactic Acid | 0.01% | (part of a blend) |
| Lecithin | 0.1% | (part of a blend) |
| Magnesium Ascorbyl Phosphate | 0.2% | |
| Methylparaben | 0.20% | |
| Morus Bombycis Root Extract | 0.01% | (part of a blend) |
| Nasturtium Officinale Extract | 5.0% | (part of a blend) |
| Palmitoyl Pentapeptide-3 | 3.0% | (part of a blend) |
| Panax Ginseng Root Extract | 5.0% | (part of a blend) |
| Panthenol | 0.1% | (part of a blend) |
| PEG-4 | 0.01% | (part of a blend) |
| PEG-100 Stearate | 4% | (part of a blend) |
| Phosphatidyl Choline | 0.1% | |
| Phospholipids | 0.5% | (part of a blend) |
| Phytonadione (Vitamin K) | 0.01% | |
| Polyamino Sugar Condensate | 5.0% | (part of a blend) |
| Polysorbate 20 | 3.0% | (part of a blend) |
| Propylparaben | 0.10% | |
| Propylene Glycol Dicaprylate/Dicaprate | 4.0% | |
| Retinyl Palmitate | 0.50% | (part of a blend) |
| Rosmarinus Officinalis (Rosemary) | .010% | |
| Leaf Extract | | |
| Sesamum Indicum (Sesame) Seed Oil | 4.0% | |
| Sodium PCA | 5.0% | |
| Sorbitan Stearate | 1% | |
| Squalane | 3.0% | |
| Stearic Acid | 2.0% | |
| Symphytum Officinale Extract | 5.0% | (part of a blend) |
| Thioctic Acid | 0.010% | |
| Tocopheryl Acetate | 0.5% | (part of a blend) |
| Triethanolamine | 0.14% | |
| Trifolium Pratense (Clover) | 5.0% | (part of a blend) |
| Flower Extract | | |
| Trisodium EDTA | 0.1% | |
| Urea | 5.0% | (part of a blend) |
| Water | 57.96% | |
| Xanthan Gum | 0.100% | |

Product 5: Day Cream (SPF 8):

The sunscreen SPF 8 used to describe this product is not intended to limit other SPF ratings. For example, SPF ratings from 2 to 80 are envisioned, although the SPF rating of 8 is preferred for this product. The sunscreen agents preferably include Octinoxate comprising 1.5% to 17% of the total weight of the Day Cream, and Oxybenzone, within the range of 0.5% to 8% of the weight of the Day Cream. This is not intended, however, to exclude PABA and other sunscreen agents that are currently known or yet to be discovered for use in conjunction with the present invention. The primary ingredients of the SPF 8 Day Cream further includes lipid nanospheres with gamma linoleic acid (GLA) derived from borage oil, including vitamins and/or esters of vitamins A, C and E dissolved in the GLA, the borage oil preferably falling within the range of 0.02% to 3.5% of the weight of the Day Cream; vitamin C in the form of Magnesium Ascorbyl Phosphate, preferably within the range of 0.0005% to 0.5% of the weight of the Day Cream; Alpha-Lipoic Acid preferably within the range of 0.0003% to 0.2% of the weight of the Day Cream; a Bio-HydriaS complex comprising a bio-herbal blend derived from comfrey, cucumber, birch leaf, watercress, clover, ginseng and St. John's wort, the Bio-Hydria® complex preferably falling within the range of 0.5% to 17% of the total weight of the Day Cream; and LYS-THR-THR-LYS-SER pentapeptides preferably totaling from 0.7% to 6.5% of the total weight of the Day Cream.

A significant synchronicity exists between the use of the SPF sunscreen and the Magnesium Aascorbyl Phosphate. Although the primary effect of the Magnesium Ascorbyl Phosphate is to increase the production of collagen and elastin, it can also act as a skin lightening agent, leaving the skin particularly susceptible to UV light exposure and damage. By using it in conjunction with an SPF sunscreen, the skin lightening aspect of the Magnesium Ascorbyl Phosphate is balanced by the protection afforded by the SPF sunscreen.

The following is a listing for ingredients of the Day Cream:

| | | |
|---|---|---|
| Allantoin | 0.10% | |
| Aloe Barbadensis Leaf Juice | 0.3% | |
| Ascorbyl Palmitate | 0.1% | (part of a blend) |
| Betula Alba Leaf Extract | 6.25% | (part of a blend) |
| Borago Officinalis Seed Oil | 0.10% | (part of a blend) |
| Butylene Glycol | 4.0% | |
| Caprylic/Capric Triglyceride | 0.10% | (part of a blend) |
| Carbomer (TEA-Carbomer) | 0.20% | |
| Cetyl Alcohol | 0.550% | |
| Cetyl Dimethicone | 3.0% | |
| Cetyl Lactate | 0.550% | |
| Citrus Aurantium Dulcis (Orange) | 0.10% | |
| Oil/Extract | | |
| Cucumis Sativus (Cucumber) | 6.25% | (part of a blend) |
| Fruit extract | | |
| Diazolidinyl Urea | 0.4% | |
| Glycerin | 3.0% | |
| Glyceryl Stearate | 3.0% | (part of a blend) |
| Glyceryl Stearate SE | 1.0% | |
| Hypericum Perforatum Extract | 6.25% | (part of a blend) |
| Isopropyl Isostearate | 4.0% | |
| Lecithin | 0.1% | |
| Magnesium Ascorbyl Phosphate | 0.001 | (part of a blend) |
| Methylparaben | 0.2% | |
| Nasturtium Officinale Extract | 6.25% | (part of a blend) |
| Octinoxate | 6.25 | |
| Octyldodecyl Neopentanoate | 7.0% | |
| Oxybenzone | 2.0% | |
| Palmitoyl Pentapeptide-3 | 3.0% | (part of a blend) |
| Panax Ginseng Root Extract | 6.25 | (part of a blend) |
| Panthenol | 0.10% | (part of a blend) |
| PEG-100 Stearate | 3% | (part of a blend) |
| Phospholipids | 0.1% | (part of a blend) |
| Polyamino Sugar Condensate | 6.25% | (part of a blend) |
| Polysorbate 20 | 3.0% | (part of a blend) |
| Propylparaben | 0.10% | |
| Retinyl Palmitate | 0.1% | (part of a blend) |
| Sodium PCA | 6.25 | (part of a blend) |
| Sorbitan Stearate | 0.8% | |
| Stearamine | 0.1% | (part of a blend) |
| Stearic Acid | 0.550% | |
| Symphytum Officinale Extract | 6.25% | (part of a blend) |
| Thioctic Acid | 0.01% | |
| Tocopherol | 0.2% | |
| Tocopheryl Acetate | 0.1% | (part of a blend) |
| Triethanolamine | 0.2% | |
| Trifolium Pratense (Clover) | 6.25% | (part of a blend) |
| Flower Extract | | |
| Trisodium EDTA | 0.050% | |
| VP/Eicosene Copolymer | 2.0 | |
| Urea | 6.25% | (part of a blend) |
| Water | 51.447% | |

Product 6: Recover Night Cream:

The primary ingredients of the Recover Night Cream include lipid nanospheres with gamma linoleic acid (GLA) derived from borage oil, including vitamins and/or esters of vitamins A, C and E dissolved in the GLA, the borage oil preferably falling within the range of 0.05% to 4% of the weight of the Recover Night Cream; vitamin C in the form of Magnesium Ascorbyl Phosphate preferably falling within the range of 0.05% to 4% of the weight of the Recover Night Cream; Alpha-Lipoic Acid preferably falling within the range of 0.0003% to 0.2% of the weight of the Recover Night Cream; a Bio-Hydria® complex comprising a bio-herbal blend derived from comfrey, cucumber, birch leaf, watercress, clover, ginseng and St. John's wort, the Bio-Hydria® complex preferably comprising from 0.05% to 8.5% of the weight of the Recover Night Cream; LYS-THR-THR-LYS-SER pentapeptides preferably falling within the range of 0.6% to 6.5% of the total weight of the Recover Night Cream; spent (barley) grain wax preferably falling within the range of 0.15% to 12% of the weight of the Recover Night Cream; and Glycine Soja (Soybean) Protein preferably falling within the range of 0.15% to 12% of the weight of the Recover Night Cream.

The following is a listing for ingredients
of the Recover Night Cream:

| | | |
|---|---|---|
| Allantoin | 0.10% | |
| Aloe Barbadensis Leaf Juice | 0.100 | |
| Ascorbyl Palmitate | 0.050% | (part of a blend) |
| Behenyl Alcohol | 5.5% | (part of a blend) |
| Betula Alba Leaf Extract | 0.1% | |
| Bis-PEG-18 Methyl Ether | 2.0% | |
| Dimethyl Silane | | |
| Borago Officinalis Seed Oil | 0.25% | (part of a blend) |
| Butylene Glycol | 3.0% | |
| Caprylic/Capric Triglyceride | 1.50% | (part of a blend) |
| Caramel | 3.5% | |
| Carbomer (TEA-Carbomer) | 0.40% | |
| Carthamus Tinctorius (Safflower) | 2.0% | |
| seed oil | | |
| Cetyl Alcohol | 5.5% | |
| Cucumis Sativus (Cucumber) | 0.100% | |
| Fruit extract | | |
| Diazolidinyl Urea | 0.3% | |
| Dimethicone | 0.5% | |
| Glycerin | 2.0% | |
| Glyceryl Stearate | 2.0% | |
| Glycine Soja (Soybean) Protein | 1.0% | |
| Hypericum Perforatum Extract | 0.1% | |
| Lecithin | .25% | (part of a blend) |
| Lauryl Alcohol | 5.5% | (part of a blend) |
| Magnesium Ascorbyl Phosphate | 0.2% | |
| Methylparaben | 0.2% | |
| Myristyl Alcohol | 5.5% | (part of a blend) |
| Nasturtium Officinale Extract | 0.1% | |
| Palmitic Acid | 5.5% | (part of a blend) |
| Palmitoyl Pentapeptide-3 | 3.0% | (part of a blend) |
| Panthenol | 1.0% | |
| Panax Ginseng Root Extract | 0.100% | |
| Persea Gratissima (Avocado Oil) | 0.50% | |
| Persea Gratissima (Avocado Oil) | 0.50% | |
| Unsaponifiables | | |
| Phosphatidyl Choline | 0.25% | |
| Phospholipids | 0.5% | (part of a blend) |
| Polyamino Sugar Condensate | 0.1% | (part of a blend) |
| Polysorbate 20 | 3.0% | (part of a blend) |
| Propylparaben | 0.10% | |
| Prunus Amygdalus Dulcis | 4.2% | |
| (Sweet almond) oil | | |
| Retinyl Palmitate | 0.5% | (part of a blend) |
| Sesamum Indicum (Sesame) | 1.2% | |
| Seed Oil | | |
| Simmondsia Chinensis (Jojoba) | 2.0 | |
| seed oil | | |
| Sodium Hyaluronate | 1.00% | |
| Sodium PCA | 0.100% | |
| Sodium Riboflavin Phospate | 0.001% | |
| Spent Grain Wax | 1.0% | |
| Squalane | 2.0% | |
| Stearic Acid | 5.50% | |
| Symphytum Officinale Extract | 0.1% | |
| Thioctic Acid | 0.01% | |
| Tocopherol | 0.4% | |
| Tocopheryl Acetate | 0.5% | (part of a blend) |
| Triethanolamine | 1.0% | |
| Trifolium Pratense (Clover) | | |
| Flower Extract | | |
| Trisodium EDTA | 0.10% | |
| Triticum Vulgare (Wheat) | 0.050% | |
| Germ oil | | |
| Urea | 0.1% | (part of a blend) |
| Water | 45.361% | |

The Daily Ten Step Regimen

The present invention envisions applying the referenced products in a daily ten-step regimen calculated to maximize skin health, including a five step morning regimen and a five step evening regimen.

Morning Regimen:

Step 1. Apply the Hydrating Cleanser (product 1) by applying a small amount of the cleanser on moist skin and massaging gently in circular motions. Excess cleanser is rinsed off after application.

Step 2. After application of the Hydrating Cleanser, apply the Toner (Product 2) directly to the face by misting and then patting dry with a cloth or misting onto a cotton ball and then smoothing the Toner over one's face.

Step 3. After application of the Toner, apply the Reactivating Facial Serum (Product 3) liberally onto skin and massage in a circular motion. The Serum is not rinsed off or wiped off after application, but remains on the face.

Step 4. After application of the Reactivating Facial Serum, apply Corrective Eye Cream (Product 4) using ring finger. Dot around eyes and gently pat until product is absorbed. The eye cream is not rinsed off after application.

Step 5: After application of the Corrective Eye Cream, apply Reality SPF8 Day Creme (Product 5), massaging liberally into the skin using circular motions. The Day Cream is not rinsed off after application, but remains on the face all day.

Evening Regimen:

Step 1. Apply the Hydrating Cleanser (Product 1) by applying a small amount of the cleanser on moist skin and massaging gently in circular motions. Excess cleanser is rinsed off after application.

Step 2. After application of the Hydrating Cleanser, apply the Toner (Product 2) directly to the face by misting and then patting dry with a cloth or misting onto a cotton ball and then smoothing the Toner over one's face.

Step 3. After application of the Toner, apply the Reactivating Facial Serum (Product 3) liberally onto skin and massage in a circular motion. The Serum is not rinsed off or wiped off after application, but remains on the face.

Step 4. After application of the Reactivating Facial Serum, apply Corrective Eye Cream (Product 4) using ring finger. Dot around eyes and gently pat until product is absorbed. The eye cream is not rinsed off after application.

Step 5: After application of the Corrective Eye Cream, apply Recover Night Creme (Product 6), massaging liberally into the skin using circular motions. The Recover Night Cream is not rinsed off after application. The user goes to bed and the night cream remains on the face throughout the night. It is washed off according to the morning regimen as described above.

A particular advantage of this combined ten-step process is that it targets unique needs through separate steps, thereby maximizing the effectiveness of the process. For example, the use of the toner-astringent in the second step includes the effect of skin tightening. If this were used prior to, or simultaneous to, the step of applying the moisturizing cleanser, the pores in the face would not be as readily cleansed. Similarly, many of the ingredients subsequent to the toner would not be as effective. Similarly, the elimination of “buckle” fat around the cheeks is not a goal of the present invention, whereas, the lipolysis of fat deposits under the eyes is a goal. Accordingly, the ingredients in the eye cream are distinct, and their application must be distinct, from the application of the reactivating facial serum. Similarly, the goals of the day cream (including sunscreen) are distinct from the goals of the night cream, which is a “recovery” and rejuvenation period.

Unlike one or two step processes of the prior art, the current invention utilizes six separate steps, including a five step morning process distinct from the five step evening process. It can, therefore, be understood that the present invention offers unique advantages over the prior art in the treatment of facial skin, and has the ability to help restore the health and vitality of facial skin in a manner superior to other products currently on the market.

Within the foregoing description, many specific details commonly understood by those skilled in the art have not been recited so as to not needlessly obscure many of the essential features of the present invention. In other instances, some non-essential details of the present invention have been recited in the detailed description to better enable the reader to make and use the claimed invention. The many details within the foregoing description are therefore not intended to limit the scope of the claims appended hereto, said claims being intended to cover alternative processes, modifications, and equivalents which may be included within the spirit and scope of the foregoing description and the appended claims.

What is claimed is:

1. A skin care regimen comprising an eye cream applied to facial skin surrounding an eye, the eye cream comprising effective amounts of pentapeptides, Alpha-Lipoic Acid and a nanosphere complex, wherein said nanosphere comprises gamma linoleic acid (GLA) within a phospholipid membrane, and a lipolysis enhancing agent for reducing fatty deposits under the eyes, including L-carnitine and coenzyme A and caffeine.

2. The skin care regimen according to claim 1 wherein said pentapeptides comprise a LYS-THR-THR-LYS-SER chemical structure.

3. The skin care regimen according to claim 1 wherein said nanosphere includes esters of vitamins A, E and C dissolved with the gamma linoleic acid.

4. The skin care regimen according to claim 1 wherein said eye cream includes magnesium ascorbyl phosphate.

5. The skin care regimen according to claim 1 wherein said eye cream includes Kojic Acid.

6. The skin care regimen according to claim 1 wherein said eye cream includes copper.

7. The skin care regimen according to claim 1 wherein said eye cream includes vitamin K.

8. The skin care regimen according to claim 1 wherein said eye cream includes a bio-herbal blend comprising members selected from a group consisting of comfrey, cucumber, birch leaf, watercress, clover, ginseng and St. John's wort.

9. The skin care regimen according to claim 1 further including a hydrating wash applied to said skin prior to application of said eye cream, the hydrating wash comprising effective amounts of:
a) nanospheres comprising gamma linoleic acid (GLA) and vitamins within a phospholipid membrane;
b) magnesium ascorbyl phosphate;
c) Alpha-Lipoic Acid; and
d) at least one cleansing agent.

10. The skin care regimen according to claim 9, wherein said cleansing agent is selected from among a group consisting of sodium lauroamphoacetate and sodium trideceth sulfate.

11. The skin care regimen according to claim 9, wherein said cleansing agent is selected from among a group consisting of disodium laureth sulfosuccinate and disodium lauroamphoacetate.

12. The skin care regimen according to claim 1 further including the application to said skin of a toner cleanser comprising effective amounts of:
a) nanospheres comprising gamma linoleic acid (GLA) and vitamins within a phospholipid membrane;
b) magnesium ascorbyl phosphate;
c) Alpha-Lipoic Acid;
d) a bio-herbal blend comprising members selected from a group consisting of comfrey, cucumber, birch leaf, watercress, clover, ginseng and St. John's wort; and
e) hamamelis virginiana (witch hazel).

13. The skin care regimen according to claim 1 further including the application to said skin of a serum comprising effective amounts of:
a) nanospheres comprising gamma linoleic acid (GLA) and vitamins within a phospholipid membrane;
b) magnesium ascorbyl phosphate;
c) Alpha-Lipoic Acid;
d) a bio-herbal blend comprising members selected from a group consisting of comfrey, cucumber, birch leaf, watercress, clover, ginseng and St. John's wort;
e) Alpha Hydroxy Acids; and
f) Beta Hydroxy Acid.

14. The skin care regimen according to claim 13 wherein said Alpha Hydroxy Acids are selected from among a group consisting of citrus medica limonum (lemon fruit extract), lactic acid, pyrus malus (apple fruit extract), saccharum officinarum (sugar cane extract), sodium lactate and green tea extract.

15. The skin regimen according to claim 13 wherein said Beta Hydroxy Acid is salicylic acid.

16. The skin care regimen according to claim 1 further including the application of a day cream to said skin comprising effective amounts of:
a) pentapeptides;
b) Alpha-Lipoic Acid;
c) a bio-herbal blend comprising members selected from a group consisting of comfrey, cucumber, birch leaf, watercress, clover, ginseng and St. John's wort;
d) nanospheres comprising gamma linoleic acid (GLA) and vitamins within a phospholipid membrane;
e) magnesium ascorbyl phosphate; and
f) a sunscreen agent.

17. The skin care regimen according to claim 16 wherein said sunscreen agent is selected from among a group consisting of octinoxate and oxybenzone.

18. The skin care regimen according to claim 1 further including the application of a night cream to said skin comprising effective amounts of:
a) pentapeptides;
b) Alpha-Lipoic Acid;
c) a bio-herbal blend comprising members selected from a group consisting of comfrey, cucumber, birch leaf, watercress, clover, ginseng and St. John's wort;
d) nanospheres comprising gamma linoleic acid (GLA) and vitamins within a phospholipid membrane;
e) magnesium ascorbyl phosphate;
f) spent grain wax; and
g) Glycine Soja (Soybean) Protein.

19. A method of treating skin comprising the step of applying an eye cream around an eye area of a face, said corrective eye cream including pentapeptides, Alpha-Lipoic Acid; magnesium ascorbyl phosphate, copper, vitamin K, a bio-herbal blend comprising members selected from a group consisting of comfrey, cucumber, birch leaf, watercress, clover, ginseng and St. John's wort, Kojic Acid, a nanosphere complex, each nanosphere comprising gamma linoleic acid (GLA) within a phospholipid membrane, and a lipolysis enhancing mixture of coenzyme A, L-carnatine and caffeine.

20. The method according to claim 19 wherein said eye cream includes Morus Bombycis Root Extract and Arctostaphylos Uva Ursi Leaf Extract.

21. The method according to claim 19 wherein said pentapeptides comprise a LYS-THR-THR-LYS-SER chemical structure.

22. The method according to claim 19 wherein said nanosphere complex includes esters of vitamins A, E and C dissolved within the gamma linoleic acid.

23. The method according to claim 19 including the step of washing said skin with a hydrating wash prior to the step of applying said eye cream, said hydrating wash comprising nanospheres that include gamma linoleic acid (GLA) and vitamins within a phospholipid membrane, magnesium ascorbyl phosphate, Alpha-Lipoic Acid, and a cleansing agent.

24. The method according to claim 23, wherein said cleansing agent includes sodium lauroamphoacetate and sodium trideceth sulfate.

25. The method according to claim 24 including the step of applying a serum to said skin subsequent to the step of applying said eye cream, said serum comprising Alpha Hydroxy Acid and Beta Hydroxy Acid.

26. The method according to claim 25 wherein said serum includes lipid nanospheres with gamma linoleic acid (GLA) derived from borage oil, including vitamin esters A, C and E dissolved in the GLA; vitamin C in the form of Magnesium Ascorbyl Phosphate; Alpha-Lipoic Acid; and a bio-herbal blend comprising plant extracts selected from among a group consisting of comfrey, cucumber, birch leaf, watercress, clover, ginseng and St. John's wort.

27. The method according to claim 26 including the step of applying to said skin a toner-astringent comprising hamamelis virginiana (witch hazel) subsequent to the step of applying said hydrating wash and prior to the step of applying said eye cream.

28. The method according to claim 27 including the step of: applying a night cream to said skin in the evening subsequent to the step of applying said eye cream, said night cream comprising Spent Grain Wax and Glycine Soja (Soybean) Protein.

29. The method according to claim 25 wherein said night cream includes a pentapeptide having a LYS-THR-THR-LYS-SER chemical structure.

30. The method according to claim 27 including the step of applying to said skin a day cream comprising at least one sunscreen agent and a pentapeptide having a LYS-THR-THR-LYS-SER chemical structure, wherein said day cream is applied in the morning subsequent to the application of said serum.

31. A method of treating facial skin and an eye area with a combination of distinct products, said products comprising a hydrating wash, a toner-astringent, an eye cream having a skin lightener, a collagen restoring agent and a lipolysis enhancing complex, a facial serum having alpha hydroxy and Beta Hydroxy Acids, a day cream having a sunscreen agent, and a night cream having spent grain wax and Glycine Soja (Soybean) Protein, wherein said products have ingredients configured to re-vitalize the production of cellular and sub-cellular facial structures including collagen, and to tighten and tone the face, the method comprising five steps performed in the morning, including the steps of:
h) washing said skin with said hydrating wash;
i) applying said toner-astringent to said skin;
j) applying said eye cream to said eye area;
k) applying said facial serum to said skin; and,
l) applying said day cream to said skin.

32. The method according to claim 31 further comprising five steps performed in the evening prior to going to bed, including the steps of:
a) washing said skin with said hydrating wash;
b) applying said toner-astringent to said skin;
c) applying said eye cream to said eye area;
d) applying said facial serum to said skin; and,
e) applying said night cream to said skin.
